(19) United States
(12) Patent Application Publication
(10) Pub. No.: US 2025/0327776 A1
(43) Pub. Date: Oct. 23, 2025

(54) INSPECTION RESULT DISPLAY DEVICE AND INSPECTION RESULT DISPLAY PROGRAM FOR TRANSDUCER ELEMENTS

(71) Applicant: FUJIFILM Corporation, Tokyo (JP)

(72) Inventor: Natsumi DANNO, Tokyo (JP)

(73) Assignee: FUJIFILM Corporation, Tokyo (JP)

(21) Appl. No.: 19/176,523

(22) Filed: Apr. 11, 2025

(30) Foreign Application Priority Data
Apr. 22, 2024 (JP) 2024-069270

(51) Int. Cl. G01N 29/44; G01N 29/30

(52) U.S. Cl. CPC G01N 29/4454; G01N 29/30

(57) ABSTRACT

A display limit value setting unit sets a display upper limit value as a display limit value based on signal intensities of a plurality of reception signals respectively corresponding to a plurality of transducer elements and acquired by a transmission and reception unit. A display control unit displays a graph indicating the signal intensity of each reception signal corresponding to each transducer element on a display. In a case where a signal intensity of a reception signal of a certain transducer element is greater than the display upper limit value, the display control unit displays the display upper limit value as the signal intensity of the reception signal on the graph. On the other hand, in a case where a signal intensity of a reception signal of a certain transducer element is equal to or less than the display upper limit value, the display control unit displays the signal intensity of the reception signal itself on the graph.

### CROSS-REFERENCE TO RELATED APPLICATIONS

This application claims benefit of priority to Japanese Patent Application No. 2024-069270 filed Apr. 22, 2024, the entire contents of which are incorporated herein by reference.

### BACKGROUND OF THE INVENTION

### 1. Field of the Invention

The present specification discloses improvements in an inspection result display device and an inspection result display program for transducer elements.

### 2. Description of the Related Art

Conventionally, an ultrasound diagnostic apparatus capable of forming an ultrasound tomographic image and performing various measurements based on reflected waves from a subject in a case where ultrasonic waves are transmitted to the subject has been known. The ultrasound diagnostic apparatus comprises an ultrasound probe that transmits and receives ultrasonic waves to and from the subject by being brought into contact with the subject. The ultrasound probe is provided with a plurality of transducer elements, and a transmission signal is supplied from an apparatus main body of the ultrasound diagnostic apparatus to each of the plurality of transducer elements, thereby transmitting ultrasonic waves from the plurality of transducer elements toward the subject. In addition, the plurality of transducer elements receive reflected waves from the subject and transmit reception signals obtained by converting the reflected waves into electrical signals to the apparatus main body.

It is common to perform an inspection before starting the use of the ultrasound diagnostic apparatus or at a predetermined timing to check whether the ultrasound probe (particularly, the plurality of transducer elements) is properly operating. Conventionally, a technique for inspecting a plurality of transducer elements has been proposed.

For example, JP2022-178120A discloses an ultrasound diagnostic apparatus having an inspection function for a plurality of transducer elements provided in an ultrasound probe, in which an ultrasonic wave is emitted from the ultrasound probe into air through airborne emission or the like to acquire a reception level of each transducer element, and an image is displayed in a bar shape extending in one direction in accordance with the arrangement of the plurality of transducer elements, with the reception level of each transducer element represented using color gradation or the like. Additionally, in the ultrasound diagnostic apparatus described in JP2022-178120A, it is possible to output a warning regarding a need for repair of the ultrasound probe based on a comparison between the reception level of each transducer element and a predetermined threshold value, or the like.

### SUMMARY OF THE INVENTION

Meanwhile, in the inspection for the plurality of transducer elements (for example, in a case where ultrasonic waves are emitted from the plurality of transducer elements into air as in JP2022-178120A), it is rare for signal values of a plurality of reception signals (for example, signal intensities of the reception signals) corresponding to the normally operating transducer elements to be identical, and it is common for the signal values of the plurality of reception signals to vary among the normally operating transducer elements.

Here, presenting the variations in the signal values of the plurality of reception signals among the transducer elements to a user may cause a problem. Since it can be determined that the transducer element is normal, for example, in a case where a signal value of a reception signal of a certain transducer element satisfies a predetermined condition (for example, it is equal to or greater than a predetermined threshold value), slight variations in the signal values of the plurality of reception signals among the transducer elements often do not pose a problem. However, presenting the variations in the signal values of the plurality of reception signals among the transducer elements to the user may cause concern, as the user who has checked the variations may worry that an abnormality has occurred in one of the plurality of transducer elements, even in a case where all of the plurality of transducer elements with varying signal values of the plurality of reception signals can be determined to be normal.

An object of an inspection result display device for transducer elements disclosed in the present specification is to reduce display of variations in signal values among a plurality of transducer elements, which are obtained from reception signals received by the transducer elements, on a display unit, in a signal value region indicating no problems with the transducer elements, in a case where the signal values are displayed as inspection results for the transducer elements.

The inspection result display device for transducer elements disclosed in the present specification is an inspection result display device for transducer elements, comprising: a reception signal acquisition unit that acquires a plurality of reception signals respectively corresponding to a plurality of transducer elements provided in an ultrasound probe, the plurality of reception signals being obtained through transmission and reception of ultrasonic waves by the plurality of transducer elements for inspecting the plurality of transducer elements; a display limit value setting unit that sets a display limit value based on a plurality of signal values respectively corresponding to the plurality of transducer elements, the plurality of signal values being obtained from the plurality of reception signals; and a display control unit that displays the plurality of signal values on a display unit in an aspect in which a correspondence relationship with the plurality of transducer elements is known, the display control unit displaying, in a case where there is a signal value exceeding the display limit value, the display limit value as the signal value.

The signal value may be a value indicating a signal intensity of a reflected wave from a predetermined depth.

The signal value may be a value indicating any one of a frequency, a phase, or a variance of a Doppler signal.

The display limit value setting unit may set the display limit value based on a parameter indicating variations in the plurality of signal values.

A determination threshold value setting unit that sets a determination threshold value based on the plurality of signal values, and a determination unit that performs a determination of whether the plurality of transducer elements are good or defective, based on a comparison between each of the plurality of signal values and the determination threshold value, may be further provided.

A memory that stores an inspection result database, the inspection result database accumulating and storing inspection results that indicate the correspondence relationship between the plurality of transducer elements and the plurality of signal values and that indicate, in a case where there is a signal value exceeding the display limit value, the display limit value as the signal value, in association with a probe ID for uniquely identifying the ultrasound probe, may be further provided, and the display control unit may display a past inspection result for the ultrasound probe, which is stored in the inspection result database, on the display unit.

In addition, an inspection result display program for transducer elements disclosed in the present specification is an inspection result display program for transducer elements, the program causing a computer to function as: a reception signal acquisition unit that acquires a plurality of reception signals respectively corresponding to a plurality of transducer elements provided in an ultrasound probe, the plurality of reception signals being obtained through transmission and reception of ultrasonic waves by the plurality of transducer elements for inspecting the plurality of transducer elements; a display limit value setting unit that sets a display limit value based on a plurality of signal values respectively corresponding to the plurality of transducer elements, the plurality of signal values being obtained from the plurality of reception signals; and a display control unit that displays the plurality of signal values on a display unit in an aspect in which a correspondence relationship with the plurality of transducer elements is known, the display control unit displaying, in a case where there is a signal value exceeding the display limit value, the display limit value as the signal value.

With the inspection result display device for transducer elements disclosed in the present specification, it is possible to reduce display of variations in signal values among a plurality of transducer elements, which are obtained from reception signals received by the transducer elements, on a display unit, in a signal value region indicating no problems with the transducer elements, in a case where the signal values are displayed as inspection results for the transducer elements.

### BRIEF DESCRIPTION OF THE DRAWINGS

FIG. 1 is a schematic diagram of a configuration of an ultrasound diagnostic apparatus according to a basic embodiment.

FIG. 2 is a conceptual diagram showing signal intensities of a plurality of reception signals corresponding to a plurality of transducer elements.

FIG. 3 is a diagram showing an example of an inspection result screen.

FIG. 4 is a diagram showing a display example of an inspection result history.

FIG. 5 is a schematic diagram of a configuration of an ultrasound diagnostic apparatus according to a modified embodiment.

FIG. 6 is a conceptual diagram showing frequencies of a plurality of Doppler signals corresponding to the plurality of transducer elements.

FIG. 7 is a conceptual diagram showing phases of the plurality of Doppler signals corresponding to the plurality of transducer elements.

FIG. 8 is a conceptual diagram showing variances of the plurality of Doppler signals corresponding to the plurality of transducer elements.

FIG. 9 is a conceptual diagram showing frequency characteristics of a frequency of a Doppler signal of a certain transducer element.

FIG. 10 is a diagram showing an example of a plot result of each transducer element in a two-dimensional space of a center frequency and a peak signal intensity.

### DESCRIPTION OF THE PREFERRED EMBODIMENTS

### Basic Embodiment

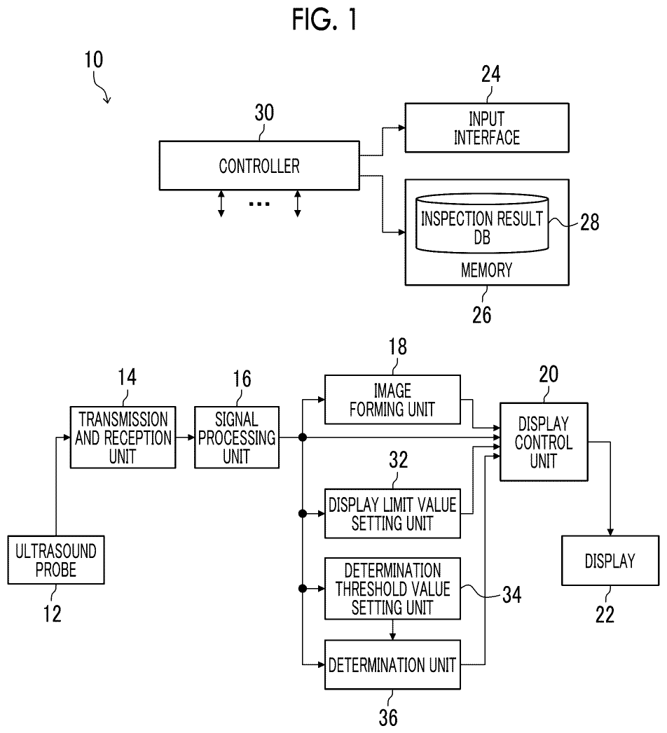

FIG. 1 is a schematic diagram of a configuration of an ultrasound diagnostic apparatus 10 as an inspection result display device for transducer elements according to a basic embodiment. The ultrasound diagnostic apparatus 10 is a medical apparatus installed in medical institutions such as a hospital.

The ultrasound diagnostic apparatus 10 is an apparatus that scans a subject with an ultrasound beam to generate an ultrasound image as a medical image or to perform various measurements based on a reception signal obtained through the scanning. The ultrasound diagnostic apparatus 10 has an inspection function of inspecting an ultrasound probe 12, which will be described below, particularly, a plurality of transducer elements provided in the ultrasound probe 12.

A transmission and reception unit 14, a signal processing unit 16, an image forming unit 18, a display control unit 20, a display limit value setting unit 32, a determination threshold value setting unit 34, and a determination unit 36, which are provided in the ultrasound diagnostic apparatus 10, are configured using a processor. The processor includes at least one of a general-purpose processing device (for example, a central processing unit (CPU) or the like) or a dedicated processing device (for example, a graphics processing unit (GPU), an application-specific integrated circuit (ASIC), a field-programmable gate array (FPGA), a programmable logic device, or the like). The processor may be configured using a plurality of processing devices that are present at physically separated positions and that collaborate with each other, rather than being configured using a single processing device. Additionally, each of the above-described units may be implemented through collaboration of hardware, such as a processor, and software.

The ultrasound probe 12 is a device that performs transmission and reception of ultrasonic waves with respect to the subject. The ultrasound probe 12 includes the plurality of transducer elements, each of which performs transmission and reception of ultrasonic waves with respect to the subject.

The transmission and reception unit 14 transmits a transmission signal to the ultrasound probe 12 (specifically, each transducer element) under the control of a controller 30 (which will be described below). Consequently, the ultrasonic waves are transmitted from each transducer element toward the subject. In addition, the transmission and reception unit 14 receives a reflected wave from the subject and converts the reflected wave into an electrical signal to form a reception signal. As a result, the transmission and reception unit 14 acquires a plurality of reception signals respectively corresponding to the plurality of transducer elements.

In particular, in the present embodiment, the transmission and reception unit 14 causes the plurality of transducer elements to transmit and receive ultrasonic waves in order to inspect the plurality of transducer elements provided in the ultrasound probe 12. For example, the transmission and reception unit 14 emits ultrasonic waves from the plurality of transducer elements into air and acquires the plurality of reception signals respectively corresponding to the plurality of transducer elements, in order to inspect the plurality of transducer elements. In this way, the transmission and reception unit 14 corresponds to a reception signal acquisition unit.

The signal processing unit 16 executes various types of processing on the reception signal formed by the transmission and reception unit 14. For example, the signal processing unit 16 performs phase alignment and addition processing of aligning and adding phases of the reception signals from the transducer elements. Consequently, a reception beam signal is formed in which information indicating signal intensities of the reflected waves from the subject is arranged in a depth direction of the subject. Additionally, the signal processing unit 16 executes various types of signal processing such as various types of filtering, detection processing, and logarithmic compression processing.

The image forming unit 18 forms an ultrasound tomographic image (B-mode image) based on the reception beam signals that have undergone the signal processing by the signal processing unit 16.

The display control unit 20 performs control to display the ultrasound tomographic image formed by the image forming unit 18 on a display 22. Additionally, the display control unit 20 displays inspection results for the plurality of transducer elements on the display 22. The display control unit 20 displays signal values of the plurality of reception signals on the display 22 in an aspect in which a correspondence relationship with the plurality of transducer elements is known, as the inspection results for the plurality of transducer elements. Details of the display aspect of the inspection results for the plurality of transducer elements will be described below.

The display 22 as a display unit is, for example, a display device configured using a liquid-crystal display, an organic electroluminescence (EL), or the like.

An input interface 24 is configured using, for example, a button, a trackball, a touch panel, and the like. The input interface 24 is used to input a user command to the ultrasound diagnostic apparatus 10.

A memory 26 includes a hard disk drive (HDD), a solid-state drive (SSD), an embedded MultiMediaCard (eMMC), a read-only memory (ROM), a random-access memory (RAM), or the like. The memory 26 stores an inspection result display program for transducer elements for operating each unit of the ultrasound diagnostic apparatus 10. The inspection result display program can also be stored in, for example, a computer-readable non-transitory storage medium such as a Universal Serial Bus (USB) memory or a CD-ROM. The ultrasound diagnostic apparatus 10 can read and execute the inspection result display program from such a storage medium.

In addition, as shown in FIG. 1, an inspection result database (DB) 28 is stored in the memory 26. The inspection result DB 28 accumulates and stores past inspection results for the ultrasound probe 12 in the ultrasound diagnostic apparatus 10. Details of the inspection result DB 28 will be described below.

The controller 30 includes at least one of a general-purpose processor (for example, a CPU or the like) or a dedicated processor (for example, a GPU, an ASIC, an FPGA, a programmable logic device, or the like). The controller 30 may be configured using a plurality of processing devices that are present at physically separated positions and that collaborate with each other, rather than being configured using a single processing device. The controller 30 controls each unit of the ultrasound diagnostic apparatus 10 in accordance with the inspection result display program stored in the memory 26.

The display limit value setting unit 32 sets a display limit value based on a plurality of signal values respectively corresponding to the plurality of transducer elements, which are obtained from the plurality of reception signals respectively corresponding to the plurality of transducer elements and acquired by the transmission and reception unit 14. The display limit value is a display limit value related to the signal value of each transducer element in a case where the display control unit 20 displays the inspection results for the plurality of transducer elements on the display 22.

In the present embodiment, a value indicating the signal intensity of the reflected wave from a predetermined depth, that is, the signal intensity of the reception signal corresponding to the predetermined depth (since the signal intensity is converted into a brightness value through imaging, the signal intensity may be referred to as a brightness value), is used as the signal value. The predetermined depth may be determined in advance, for example, 1 cm, 4 cm, or the like. The signal intensity of the reception signal in the following description means the signal intensity of the reception signal corresponding to the predetermined depth. Based on this, the display limit value setting unit 32 sets a display upper limit value as the display limit value based on the signal intensities of the plurality of reception signals respectively corresponding to the plurality of transducer elements. The signal intensities of the plurality of reception signals may be signal intensities of the reception signals before the signal processing by the signal processing unit 16, may be signal intensities of the reception signals after the signal processing by the signal processing unit 16, or may be signal intensities (brightness values) in ultrasound image data formed by the image forming unit 18.

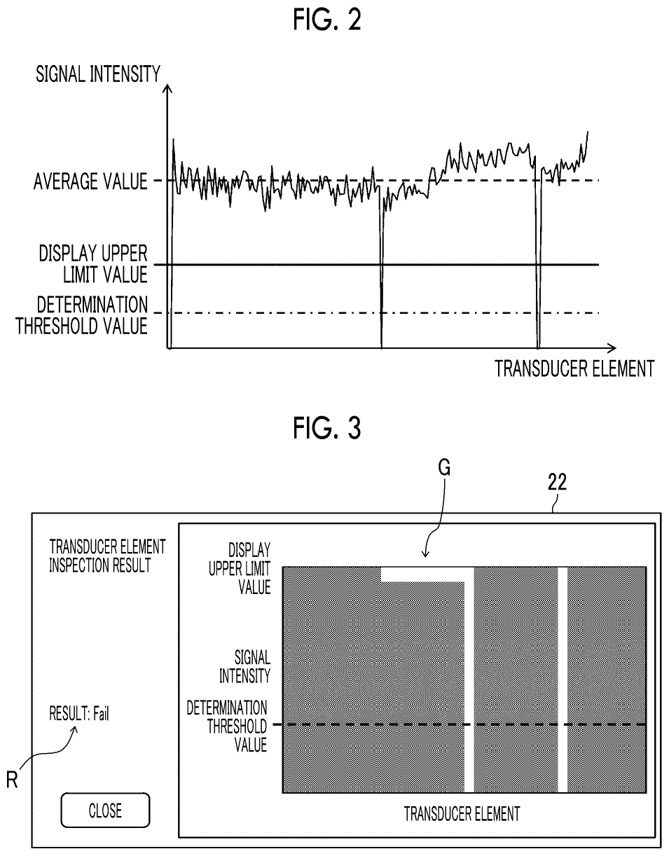

FIG. 2 is a conceptual diagram showing the signal intensities of the plurality of reception signals corresponding to the plurality of transducer elements. Specifically, the display limit value setting unit 32 sets the display upper limit value based on a representative value of the signal intensities of the plurality of reception signals. In the present embodiment, an average value is used as the representative value, but the representative value may be, for example, a median value, a mode value, or the like. For example, the display limit value setting unit 32 sets X % of the representative value of the signal intensities of the plurality of reception signals as the display upper limit value. Here, X may be set in advance by a program designer or the like of the inspection result display device for transducer elements and is set to, for example, a value such as 20 to 80. In the example of FIG. 2, the display upper limit value is set to 50% of the average value of the signal intensities of the plurality of reception signals (that is, X=50).

Additionally, the display limit value setting unit 32 may further set the display limit value based on a parameter indicating variations in the signal intensities of the plurality of reception signals. The parameter indicating the variations is a concept including a variance, a standard deviation, or the like of the signal intensities of the plurality of reception signals.

For example, the display limit value setting unit 32 may calculate a variance a of the signal intensities of the plurality of reception signals and set a value obtained by subtracting a value based on the variance a from a representative value of the signal intensities of the plurality of reception signals as the display upper limit value. For example, a value of ((average value of signal intensities of plurality of reception signals)−βα) is set as the display upper limit value. Here, β may be set in advance by the program designer or the like of the inspection result display device for transducer elements and is set to, for example, a value such as 1 to 5.

In addition, for example, the display limit value setting unit 32 obtains the standard deviation of the signal intensities of the plurality of reception signals and sets a value of ((average value of signal intensities of plurality of reception signals)−α×(standard deviation)) as the display upper limit value. Here, α may also be set in advance by the program designer or the like of the inspection result display device for transducer elements.

The display control unit 20 displays the inspection results for the plurality of transducer elements on the display 22 based on the display limit value set by the display limit value setting unit 32. FIG. 3 is a diagram showing an example of an inspection result screen showing the inspection results for the plurality of transducer elements. On the inspection result screen shown in FIG. 3, a graph G shows the signal intensity of each reception signal corresponding to each transducer element. The graph G is a graph in which a horizontal axis represents the transducer elements and a vertical axis represents the signal intensities.

On the graph G, in a case where a signal intensity of a reception signal of a certain transducer element exceeds the display limit value, the display control unit 20 displays the display limit value as the signal intensity of the reception signal. In the present embodiment, since the display limit value is the display upper limit value, the display control unit 20 displays the display upper limit value as the signal intensity of the reception signal in a case where the signal intensity of the reception signal of a certain transducer element is greater than the display upper limit value. On the other hand, in a case where the signal intensity of the reception signal of a certain transducer element is equal to or less than the display upper limit value, the display control unit 20 displays the signal intensity of the reception signal itself. For example, in a case where the display upper limit value is x, the display control unit 20 displays the signal intensity of the reception signal of the transducer element as x on the graph G even in a case where the signal intensity of the reception signal of a certain transducer element is equal to or greater than x. On the other hand, in a case where the signal intensity of the reception signal of a certain transducer element is equal to or less than x, the display control unit 20 displays the signal intensity of the reception signal of the transducer element on the graph G as a value equal to or less than x. In other words, the display control unit 20 does not represent the signal intensity equal to or greater than the display upper limit value on the graph G and displays all those signal intensities as the display upper limit value.

Consequently, even in a case where the signal intensities of the reception signals of the plurality of transducer elements vary in a value region equal to or greater than the display upper limit value (refer to FIG. 2), the signal intensities of the reception signals of the plurality of transducer elements are displayed as the display upper limit value on the graph G. That is, the user is not notified of the variations in the signal intensities of the reception signals of the plurality of transducer elements in the value region equal to or greater than the display upper limit value. As a result, it is possible to reduce unnecessary concern felt by the user.

Description returns to FIG. 1. The determination threshold value setting unit 34 sets a determination threshold value based on the signal values of the plurality of reception signals respectively corresponding to the plurality of transducer elements and acquired by the transmission and reception unit 14. The determination threshold value is a threshold value used in a case where the determination unit 36, which will be described below, performs a determination of whether the plurality of transducer elements are good or defective, based on the signal values of the plurality of reception signals.

Similar to the display limit value setting unit 32, in the present embodiment, the determination threshold value setting unit 34 sets the determination threshold value based on the signal intensities of the plurality of reception signals respectively corresponding to the plurality of transducer elements (refer to FIG. 2). Specifically, the determination threshold value setting unit 34 sets the determination threshold value based on the representative value of the signal intensities of the plurality of reception signals. In the present embodiment, the average value is used as the representative value, but the representative value may be, for example, a median value, a mode value, or the like. For example, the determination threshold value setting unit 34 sets Y % of the representative value of the signal intensities of the plurality of reception signals as the determination threshold value. Here, Y may be set in advance by the program designer or the like of the inspection result display device for transducer elements. In the example of FIG. 2, the determination threshold value is set to 20% of the average value of the signal intensities of the plurality of reception signals (that is, Y=20).

Additionally, the determination threshold value setting unit 34 may further set the determination threshold value based on the parameter (variance or standard deviation) indicating the variations in the signal intensities of the plurality of reception signals, similar to the display limit value setting unit 32.

The determination unit 36 performs a determination of whether the plurality of transducer elements are good or defective, based on a comparison between each of the signal values of the plurality of reception signals and the determination threshold value set by the determination threshold value setting unit 34. Specifically, in a case where all the signal intensities of the plurality of reception signals are equal to or greater than the determination threshold value, the determination unit 36 determines that the plurality of transducer elements are good (normal). On the other hand, in a case where any of the signal intensities of the plurality of reception signals is less than the determination threshold value, the determination unit 36 determines that the plurality of transducer elements are defective (abnormal).

The display control unit 20 may also display a determination result R of the determination unit 36 on the inspection result screen (refer to FIG. 3). In the example of FIG. 3, since there is a transducer element of which the signal value is less than the determination threshold value, “Fail” meaning a defective condition is displayed as the determination result R of the determination unit 36.

The inspection results for the plurality of transducer elements may be accumulated and stored in the inspection result DB 28. In the present embodiment, the inspection results (that is, information corresponding to the graph G in FIG. 3) that indicate the correspondence relationship between the plurality of transducer elements and the signal values of the plurality of reception signals and that indicate, in a case where there is a signal value equal to or greater than the display upper limit value, the display upper limit value as the signal value are associated with a probe ID for uniquely identifying the ultrasound probe 12 including the plurality of transducer elements to be inspected and are stored as the inspection result history in the inspection result DB 28. The probe ID may be stored in a memory of the ultrasound probe 12, and an apparatus main body of the ultrasound diagnostic apparatus 10 can acquire the probe ID from the connected ultrasound probe 12. Additionally, the inspection result and the probe ID may be further associated with the inspection date and time or the determination result by the determination unit 36 and stored in the inspection result DB 28 as the inspection result history.

The display control unit 20 may display the past inspection results for the ultrasound probe 12, which are stored in the inspection result DB 28, on the display 22. Specifically, the display control unit 20 acquires the probe ID from the ultrasound probe 12 connected to the apparatus main body of the ultrasound diagnostic apparatus 10 and extracts the inspection result history having the acquired probe ID from the inspection result DB 28. Then, the display control unit 20 displays the inspection result history extracted from the inspection result DB 28 on the display 22.

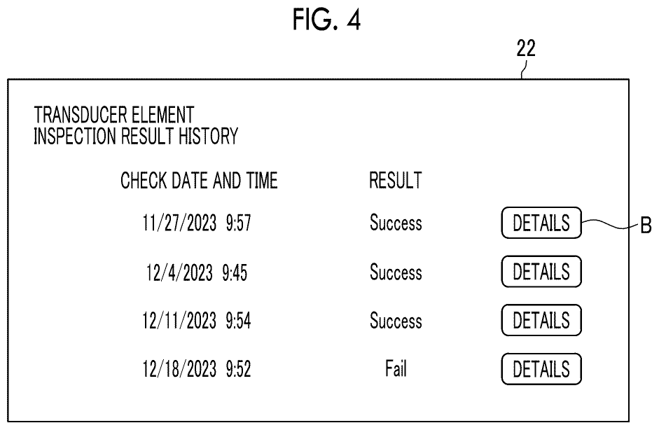

FIG. 4 is a diagram showing a display example of the inspection result history. As shown in FIG. 4, in the present embodiment, the display control unit 20 displays a list of combinations of the inspection date and time and the determination result among the inspection result histories extracted from the inspection result DB 28. Consequently, the user can grasp the determination result in the past for the ultrasound probe 12 at a glance. In addition, the display control unit 20 displays a detail button B in association with the inspection date and time and the determination result. In a case where the user selects the detail button B, the display control unit 20 displays the inspection result (that is, the graph G (refer to FIG. 3)) corresponding to the inspection date and time and the determination result associated with the detail button B on the display 22. As a result, the user can grasp the signal value of each transducer element in the past.

### Modified Embodiment

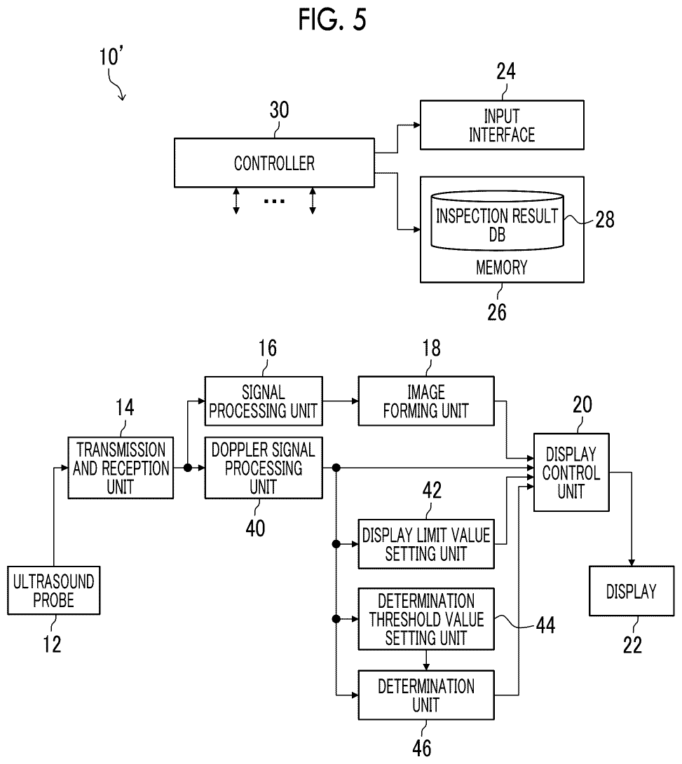

FIG. 5 is a schematic diagram of a configuration of an ultrasound diagnostic apparatus 10′ as an inspection result display device for transducer elements according to a modified embodiment. In the ultrasound diagnostic apparatus 10′ according to the modified embodiment, the components that exhibit the same functions as the ultrasound diagnostic apparatus 10 according to the basic embodiment shown in FIG. 1 are denoted by the same reference numerals as those in FIG. 1, and the description thereof will be omitted. In the ultrasound diagnostic apparatus 10 according to the basic embodiment, the signal intensities of the reception signals of the transducer elements are displayed as the inspection results for the plurality of transducer elements of the ultrasound probe 12, the display upper limit value is set, the determination threshold value is set, and a determination of whether the plurality of transducer elements are good or defective is performed, based on the signal intensity of the reception signal of each transducer element. In the ultrasound diagnostic apparatus 10′ according to the modified embodiment, the display upper limit value is set, the determination threshold value is set, and a determination of whether the plurality of transducer elements are good or defective is performed, based on a Doppler signal corresponding to each transducer element, which is obtained from the reception signal of each transducer element.

A Doppler signal processing unit 40 executes quadrature detection processing of separating the reception signal from the transmission and reception unit 14 into a complex signal (a real part signal (I component) and an imaginary part signal (Q component)) on the reception signal, filtering of applying a wall filter for removing noise (clutter components) caused by body movement or the like of the subject to the complex signal obtained by the quadrature detection processing, autocorrelation calculation of calculating a correlation between the I component and the Q component, and the like, thereby obtaining the Doppler signal. The Doppler signal is a signal indicating a frequency (a shift frequency (a difference between a transmission frequency and a reception frequency)), a phase, and a variance of a velocity component (that is, a shift frequency) for each reception signal corresponding to each transducer element.

A display limit value setting unit 42 sets the display limit value based on the signal values of a plurality of Doppler signals respectively corresponding to the plurality of transducer elements and acquired by the Doppler signal processing unit 40 based on the plurality of reception signals.

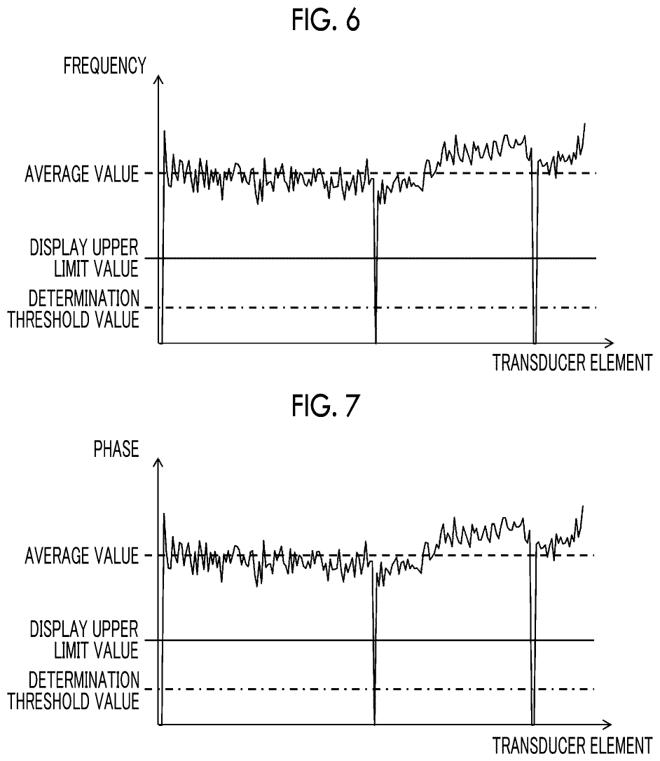

FIG. 6 is a conceptual diagram showing frequencies of the plurality of Doppler signals corresponding to the plurality of transducer elements. The display limit value setting unit 42 can set the display upper limit value as the display limit value based on the frequency as the signal value of the Doppler signal. For example, the display limit value setting unit 32 sets the display upper limit value based on the representative value (in the present embodiment, the average value) of a plurality of frequencies corresponding to the plurality of transducer elements. For example, the display limit value setting unit 32 sets X % (X is, for example, 20 to 80) of the representative value of the frequencies of the plurality of Doppler signals as the display upper limit value. In the example of FIG. 6, the display upper limit value is set to 50% of the average value of the frequencies of the plurality of Doppler signals (that is, X=50).

FIG. 7 is a conceptual diagram showing phases of the plurality of Doppler signals corresponding to the plurality of transducer elements. The display limit value setting unit 42 can set the display upper limit value as the display limit value based on the phase as the signal value of the Doppler signal. For example, the display limit value setting unit 32 sets the display upper limit value based on the representative value (in the present embodiment, the average value) of a plurality of phases corresponding to the plurality of transducer elements. For example, the display limit value setting unit 32 sets X % (X is, for example, 20 to 80) of the representative value of the phases of the plurality of Doppler signals as the display upper limit value. In the example of FIG. 7, the display upper limit value is set to 50% of the average value of the phases of the plurality of Doppler signals (that is, X=50).

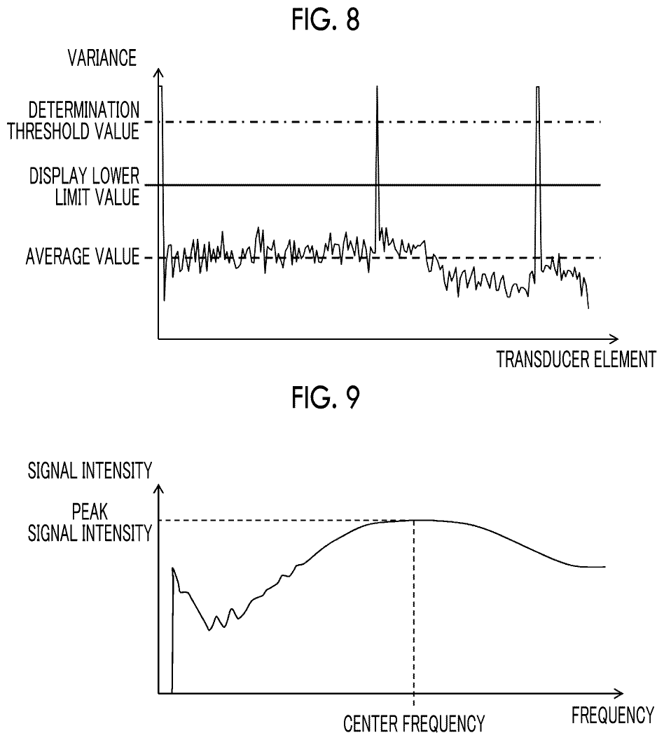

FIG. 8 is a conceptual diagram showing variances of the plurality of Doppler signals corresponding to the plurality of transducer elements. The display limit value setting unit 42 can set a display lower limit value as the display limit value based on the variance of the Doppler signal as the signal value. For example, the display limit value setting unit 32 sets the display lower limit value based on the representative value (in the present embodiment, the average value) of a plurality of variances corresponding to the plurality of transducer elements. For example, the display limit value setting unit 32 sets a value obtained by adding a predetermined value to the representative value of the variances of the plurality of Doppler signals as the display lower limit value.

Similar to the basic embodiment, the display limit value setting unit 42 may further set the display limit value based on a parameter indicating variations in the signal values of the plurality of Doppler signals.

In the modified embodiment, the display control unit 20 also displays the inspection results for the plurality of transducer elements on the display 22 based on the display limit value set by the display limit value setting unit 42. Specifically, in a case where a signal value of a Doppler signal of a certain transducer element exceeds the display limit value, the display control unit 20 displays the display limit value as the signal value of the Doppler signal. In a case of being based on the frequency or the phase of the Doppler signal, the display upper limit value as the display limit value is set. Therefore, in a case where the frequency or the phase of the Doppler signal of a certain transducer element is greater than the display upper limit value, the display control unit 20 displays the display upper limit value as the frequency or the phase of the Doppler signal. On the other hand, in a case where the frequency or the phase of the Doppler signal of a certain transducer element is equal to or less than the display upper limit value, the display control unit 20 displays the frequency or the phase of the Doppler signal itself. In a case of being based on the variance of the Doppler signal, the display lower limit value as the display limit value is set. Therefore, in a case where the variance of the Doppler signal of a certain transducer element is less than the display lower limit value, the display control unit 20 displays the display lower limit value as the variance of the Doppler signal. On the other hand, in a case where the variance of the Doppler signal of a certain transducer element is equal to or greater than the display lower limit value, the display control unit 20 displays the variance of the Doppler signal itself.

Description returns to FIG. 5. A determination threshold value setting unit 44 sets the determination threshold value based on the signal values of the plurality of Doppler signals respectively corresponding to the plurality of transducer elements and acquired by the Doppler signal processing unit 40 based on the plurality of reception signals.

Similar to the display limit value setting unit 42, in the present embodiment, the determination threshold value setting unit 44 sets the determination threshold value based on the signal values of the plurality of Doppler signals respectively corresponding to the plurality of transducer elements (refer to FIGS. 6 to 8). Specifically, the determination threshold value setting unit 44 sets the determination threshold value based on the representative value (for example, the average value) of the signal values of the plurality of Doppler signals. For example, the determination threshold value setting unit 44 sets Y % (Y is, for example, 20) of the representative value of the signal values of the plurality of Doppler signals as the determination threshold value.

Additionally, the determination threshold value setting unit 44 may further set the determination threshold value based on the parameter (variance or standard deviation) indicating the variations in the signal values of the plurality of Doppler signals, similar to the display limit value setting unit 42.

A determination unit 46 performs a determination of whether the plurality of transducer elements are good or defective, based on a comparison between each of the signal values of the plurality of Doppler signals and the determination threshold value set by the determination threshold value setting unit 44. Specifically, in a case where all of the frequencies or all of the phases of the plurality of Doppler signals are equal to or greater than the determination threshold value, the determination unit 46 determines that the plurality of transducer elements are good (normal). On the other hand, in a case where any of the frequencies or any of the phases of the plurality of Doppler signals is less than the determination threshold value, the determination unit 46 determines that the plurality of transducer elements are defective (abnormal). In addition, in a case where all of the variances of the plurality of Doppler signals are less than the determination threshold value, the determination unit 46 determines that the plurality of transducer elements are good (normal). On the other hand, in a case where any of the variances of the plurality of Doppler signals is equal to or greater than the determination threshold value, the determination unit 46 determines that the plurality of transducer elements are defective (abnormal).

Additionally, in the modified embodiment, the determination unit 46 may perform a determination of whether the plurality of transducer elements are good or defective without being based on the determination threshold value set by the determination threshold value setting unit 44. For example, the determination unit 46 may perform a determination of whether the plurality of transducer elements are good or defective, based on the frequency characteristics (refer to FIG. 9) of the frequency of the Doppler signal of each transducer element. For example, the determination unit 46 calculates the cross-correlation of the frequency characteristics of the transducer elements and determines that, in a case where there is a transducer element with a correlation coefficient equal to or less than a predetermined threshold value, the plurality of transducer elements are defective (abnormal).

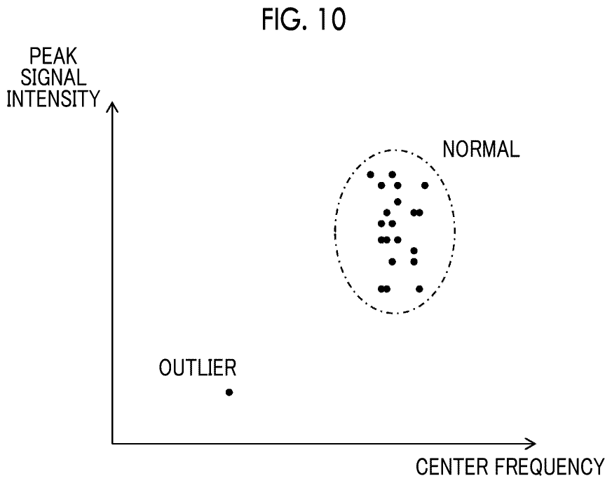

In addition, the determination unit 46 may acquire a center frequency and a peak signal intensity of the frequency characteristics of the frequency of the Doppler signal of each transducer element (refer to FIG. 9), plot each transducer element on a two-dimensional map of the center frequency and the peak signal intensity as shown in FIG. 10, and determine that the plurality of transducer elements are defective (abnormal) in a case where there is a transducer element having an outlier.

Additionally, the determination unit 46 may perform a determination of whether the plurality of transducer elements are good or defective, based on the fractional bandwidth of the frequency characteristics of the frequency of the Doppler signal of each transducer element. The fractional bandwidth is a parameter represented by ((frequency bandwidth/center frequency)×100). In a case where there is a transducer element with a fractional bandwidth less than a predetermined threshold value, it is determined that the plurality of transducer elements are defective (abnormal).

Similar to the basic embodiment, in the modified embodiment, the display control unit 20 may also display the determination result of the determination unit 46 on the inspection result screen.

Although the inspection result display device for transducer elements according to the present disclosure has been described above, the inspection result display device for transducer elements according to the present disclosure is not limited to the above-described embodiments, and various changes can be made without departing from the gist of the present disclosure.

For example, in the above-described embodiment, the inspection result display device for transducer elements is the ultrasound diagnostic apparatus 10, but the inspection result display device is not limited to the ultrasound diagnostic apparatus 10. For example, the inspection result display device may be a personal computer (PC), a server, or the like including a processor, a memory, a communication interface, an input interface, and a display. In this case, the processor of the PC or the server as the inspection result display device exerts the functions as the display limit value setting units 32 and 42, the determination threshold value setting units 34 and 44, the determination units 36 and 46, and the display control unit 20, acquires a plurality of reception signals corresponding to a plurality of transducer elements from the ultrasound diagnostic apparatus or the like, and performs the inspection for the ultrasound probe 12 (the plurality of transducer elements) based on the acquired plurality of reception signals.

What is claimed is:

1. An inspection result display device for transducer elements, comprising:
a reception signal acquisition unit that acquires a plurality of reception signals respectively corresponding to a plurality of transducer elements provided in an ultrasound probe, the plurality of reception signals being obtained through transmission and reception of ultrasonic waves by the plurality of transducer elements for inspecting the plurality of transducer elements;
a display limit value setting unit that sets a display limit value based on a plurality of signal values respectively corresponding to the plurality of transducer elements, the plurality of signal values being obtained from the plurality of reception signals; and
a display control unit that displays the plurality of signal values on a display unit in an aspect in which a correspondence relationship with the plurality of transducer elements is known, the display control unit displaying, in a case where there is a signal value exceeding the display limit value, the display limit value as the signal value.

2. The inspection result display device for transducer elements according to claim 1,
wherein the signal value is a value indicating a signal intensity of a reflected wave from a predetermined depth.

3. The inspection result display device for transducer elements according to claim 1,
wherein the signal value is a value indicating any one of a frequency, a phase, or a variance of a Doppler signal.

4. The inspection result display device for transducer elements according to claim 1,
wherein the display limit value setting unit sets the display limit value based on a parameter indicating variations in the plurality of signal values.

5. The inspection result display device for transducer elements according to claim 1, further comprising:
a determination threshold value setting unit that sets a determination threshold value based on the plurality of signal values; and
a determination unit that performs a determination of whether the plurality of transducer elements are good or defective, based on a comparison between each of the plurality of signal values and the determination threshold value.

6. The inspection result display device for transducer elements according to claim 1, further comprising:
a memory that stores an inspection result database, the inspection result database accumulating and storing inspection results that indicate the correspondence relationship between the plurality of transducer elements and the plurality of signal values and that indicate, in a case where there is a signal value exceeding the display limit value, the display limit value as the signal value, in association with a probe ID for uniquely identifying the ultrasound probe,
wherein the display control unit displays a past inspection result for the ultrasound probe, which is stored in the inspection result database, on the display unit.

7. A non-transitory computer-readable storage medium storing an inspection result display program for transducer elements, the program causing a computer to function as:
a reception signal acquisition unit that acquires a plurality of reception signals respectively corresponding to a plurality of transducer elements provided in an ultrasound probe, the plurality of reception signals being obtained through transmission and reception of ultrasonic waves by the plurality of transducer elements for inspecting the plurality of transducer elements;
a display limit value setting unit that sets a display limit value based on a plurality of signal values respectively corresponding to the plurality of transducer elements, the plurality of signal values being obtained from the plurality of reception signals; and
a display control unit that displays the plurality of signal values on a display unit in an aspect in which a correspondence relationship with the plurality of transducer elements is known, the display control unit displaying, in a case where there is a signal value exceeding the display limit value, the display limit value as the signal value.
